(19) United States
(12) Patent Application Publication
(10) Pub. No.: US 2013/0296986 A1
(43) Pub. Date: Nov. 7, 2013

(54) Therapeutic Cold Compress Apparatus and Method for Use

(71) Applicant: Alan Lynn Dukes, Woodstock, GA (US)

(72) Inventor: Alan Lynn Dukes, Woodstock, GA (US)

(21) Appl. No.: 13/886,739

(22) Filed: May 3, 2013

Related U.S. Application Data
(60) Provisional application No. 61/643,192, filed on May 4, 2012.
(60) Provisional application No. 61/671,929, filed on Jul. 16, 2012.
(60) Provisional application No. 61/745,456, filed on Dec. 21, 2012.
(60) Provisional application No. 61/749,857, filed on Jan. 7, 2013.

(51) Int. Cl. A61F 7/10

(52) U.S. Cl. CPC A61F 7/10
USPC 607/110; 607/112; 607/111; 607/108

(57) ABSTRACT

Various embodiments are disclosed for applying a cool compression effect. One embodiment, among others, is an adjustable wrap configured as a sleeve with a variable peripheral size. The wrap comprises a first elongated layer comprising a liquid-absorbent sheet and a second elongated layer. The first elongated layer and the second elongated layer are substantially coextensive in size, and one of the first elongated layer and the second elongated layer is configured to be applied directly to a selected body part and provide a cold compression effect to the body part. The adjustable wrap further comprises a fastener coupled to one of the first elongated layer and the second elongated layer, the fastener comprising a first section and a second section for providing an adjustable peripheral size of the wrap.

### CROSS-REFERENCE TO RELATED APPLICATION

This application claims priority to, and the benefit of, U.S. Provisional Patent Application entitled, “Therapeutic Cold Wrap and Sleeve and Method for Use,” having Ser. No. 61/643,192, filed on May 4, 2012; “Therapeutic Cold Wrap and Sleeve and Method for Use,” having Ser. No. 61/671,929, filed on Jul. 16, 2012; “Therapeutic Cold Wrap and Sleeve and Method for Use,” having Ser. No. 61/745,456, filed on Dec. 21, 2012; and “Therapeutic Cold Wrap and Sleeve and Method for Use,” having Ser. No. 61/749,857, filed on Jan. 7, 2013, all of which are incorporated herein by reference in their entireties.

### BACKGROUND

Cold compression therapy seeks to reduce pain and swelling caused by an injury to soft tissues and is especially useful for sprains, strains, pulled muscles, and so on. Cold therapy generally involves applying ice or a cold pack directly to a body part for a certain duration of time. However, one perceived shortcoming with traditional methods of cold therapy is that application of ice or a cold pack is limited as the patient may experience discomfort during prolonged application. Furthermore, prolonged application of extreme cold can cause even further injury to the individual.

### SUMMARY

Briefly described, one embodiment, among others, is an adjustable wrap configured as a sleeve with a variable peripheral size. The wrap comprises a first elongated layer comprising a liquid-absorbent sheet and a second elongated layer, the first elongated layer and the second elongated layer being substantially coextensive in size, wherein one of the first elongated layer and the second elongated layer is configured to be applied directly to a selected body part and provide a cold compression effect to the body part. The wrap further comprises a fastener coupled to one of the first elongated layer and the second elongated layer, the fastener comprising a first section and a second section for providing an adjustable peripheral size of the wrap.

Another embodiment is a therapeutic apparatus that comprises a first layer comprising a liquid-absorbent sheet a second layer, the first layer and the second layer being substantially coextensive in size, wherein one of the first layer and the second layer is configured to be applied directly to a selected body part, and wherein the first layer and the second layer are configured in a shape substantially conforming with the selected body part to receive a cold compression effect.

Another embodiment is a cooling apparatus that comprises a first layer comprising a liquid-absorbent sheet and a second layer, the first layer and the second layer being substantially coextensive in size, wherein one of the first layer and the second layer is configured to be applied directly to a selected body part, and wherein the first layer and the second layer are configured in a headwear shape for applying a cold compression effect to a forehead region.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a wrap form of the product, which may generally be provided in a planar form, which can be wrapped around a body portion in accordance with various embodiments of the present disclosure.

FIG. 2 shows a sleeve form of the product in accordance with various embodiments of the present disclosure.

FIG. 3 shows an embodiment comprising an insole that can be placed inside footwear in accordance with various embodiments of the present disclosure.

FIGS. 4A and 4B show the product in the form of headwear in accordance with various embodiments of the present disclosure.

FIGS. 5A and 5B show the product in the form of a vest or reverse-halter top, wherein straps are provided in the front in accordance with various embodiments of the present disclosure.

FIG. 6 shows the product in the form of a glove, thereby providing cooling to the hand and wrist in accordance with various embodiments of the present disclosure.

FIG. 7 shows the product in the form of a sandal or flip-flop in accordance with various embodiments of the present disclosure.

FIGS. 8A and 8B show the product in a form that can readily be applied to the shin or thigh area as a wrap in accordance with various embodiments of the present disclosure.

FIG. 9 shows the product in the form of a forearm wrap in accordance with various embodiments of the present disclosure.

FIG. 10 shows the product in the form of an elbow wrap in accordance with various embodiments of the present disclosure.

FIG. 11 shows the product in the form of a shoulder wrap in accordance with various embodiments of the present disclosure.

FIGS. 12B and 12B show the product in a form suitable for a lower back wrap in accordance with various embodiments of the present disclosure.

FIG. 13 shows the product in the form of an ankle wrap in accordance with various embodiments of the present disclosure.

FIG. 14 shows the product in the form of a foot wrap in accordance with various embodiments of the present disclosure.

FIG. 15 shows the product in the form of a knee wrap in accordance with various embodiments of the present disclosure.

FIG. 16 shows the product in the form of use as a headband in accordance with various embodiments of the present disclosure.

FIGS. 17A and 17B show the product in the form of use as a neckband in accordance with various embodiments of the present disclosure.

### DETAILED DESCRIPTION

Various embodiments are directed to a therapeutic line of accessories and devices for providing cool or cold compresses for parts of a human or animal body. The accessories may be embodied in various forms including wraps, sleeves, sheets, and pads, and may be particularly shaped and sized to snugly fit different locations on a human body to maximize the cold compression effect. In a preferred embodiment, the sleeves or wraps are constructed on the outer exposure from a material known as Poly Vinyl Alcohol (PVA). However, embodiments disclosed herein are not limited to the use of PVA as other materials suitable for applying a cool or cold compress effect may be used. These product lines may be produced from any source of material that would allow for the products to work most effectively.

In accordance with various embodiments, two outer layers of PVA are heat compressed onto a mesh material in the center in a sandwich-like configuration. The interposed mesh is made from a suitable material to give strength and stability to the product. In some embodiments, the mesh may be formed from a relatively inelastic material and is combined with the layers of PVA to prevent deformation of the overall product. In other embodiments, the mesh may be formed from elastomeric material to prevent a certain degree of deformation, with sufficient memory to return the product to its original form or shape. For some embodiments, the mesh material may be omitted, leaving only the PVA material in the products and enhancing flexibility of the product. For yet other embodiments, the inner layer of the product may be constructed of tricot mesh for ease of pulling over, taking off, or wrapping around a body part.

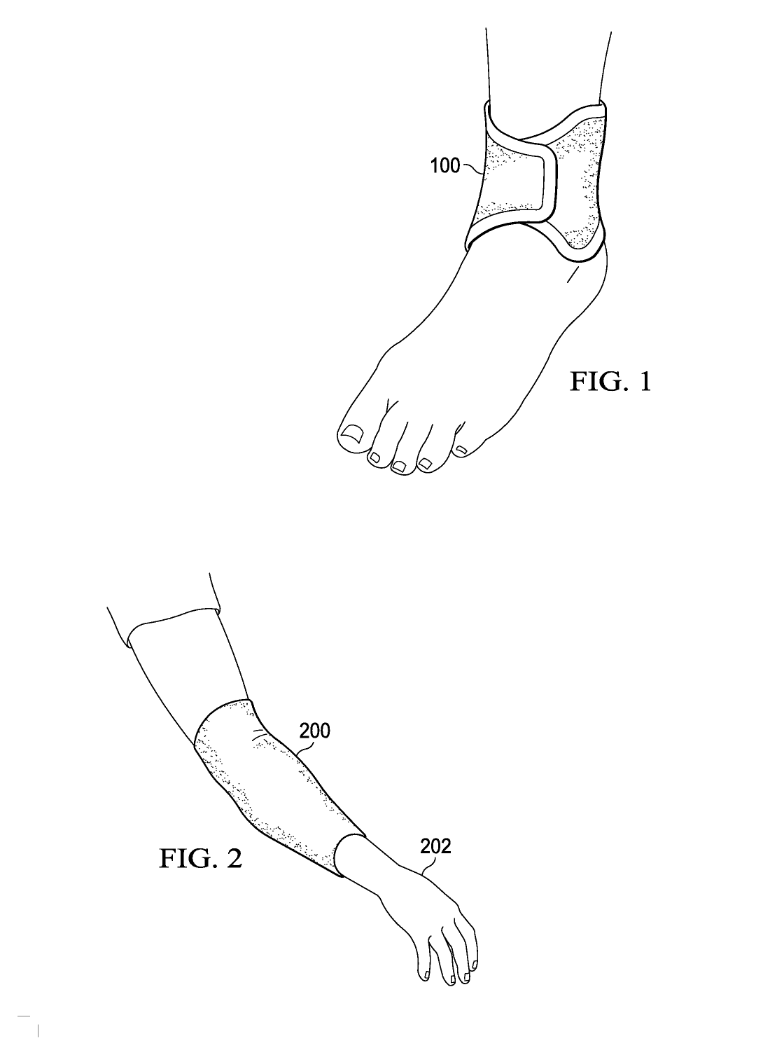

Reference is made to FIG. 1, which shows a wrap form of the product 100 in accordance with various embodiments. The product 100 may be constructed in a planar form, which can be wrapped around a body portion (e.g., an ankle). A hook-and-loop faster or another reusable adhesive may be provided on the product 100 to secure it in place until it is ready to be removed. It will be appreciated that product embodiments in this form may be readily applied to different locations of the body.

In this regard, one embodiment is a wrap that comprises a first elongated layer comprising a liquid-absorbent sheet and a second elongated layer, the first elongated layer and the second elongated layer being substantially coextensive in size, wherein one of the first elongated layer and the second elongated layer is configured to be applied directly to a selected body part and provide a cold compression effect to the body part. The wrap further comprises a fastener coupled to one of the first elongated layer and the second elongated layer, the fastener comprising a first section and a second section for providing an adjustable peripheral size of the wrap.

FIG. 2 shows a sleeve form of the product 200, where the product 200 may be stretched over a body part 202, such as a forearm or calf muscle. Preferably, the product 200 in this form is constructed with an inner mesh to provide sufficient deformation and resilience to be stretched into place and snugly and comfortably retained. As described above, the cool sleeve embodiment 200 is designed to be pulled over an injured or inflamed area of the body. Alternatively, the cool wrap may be designed to wrap an injured or inflamed area of the body with the use of hook-and-loop fasteners or other reusable adhesive materials. The areas of the body that would benefit from these aids may include, but are not limited to, the ankle, knee, back, wrist, elbow, shoulder, neck, and head.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

One embodiment, using hook-and-loop fasteners, may be constructed of two outer layers of PVA which are heat compressed onto a mesh material located in the center of each outer layer. This mesh gives stability to this particular embodiment. Another embodiment will be two layers of PVA without a mesh center. This will create a more elastic type of embodiment which will serve the purpose of sliding over various body parts. This non-meshed embodiment may also require an elastic type of material that will allow for ease of slipping over the skin while also allowing the cool sensation to reach the skin.

Whether the mesh or non-mesh embodiment is used may depend upon the shape of the product and the area of the body of intended use. For example, embodiments intended for joint areas, such as knee, elbow, and shoulder, may prefer using the mesh embodiment for additional strength and durability (due to the expected movement of the product with body movement about the joint).

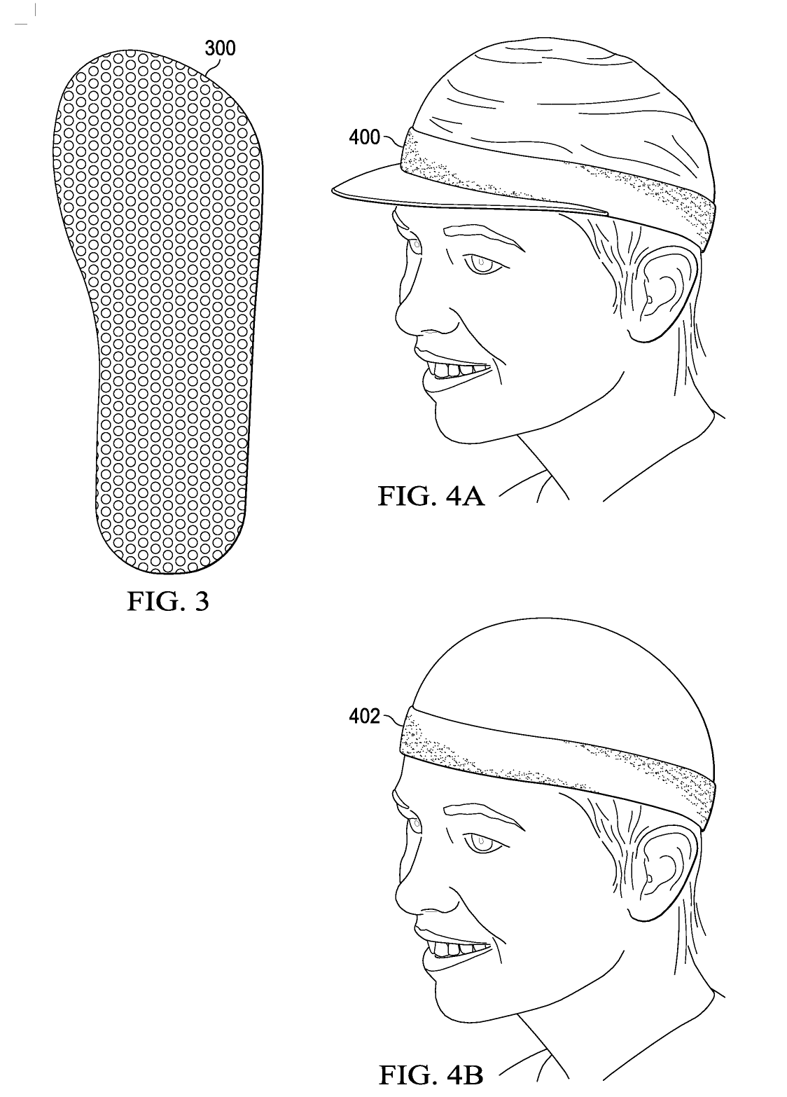

A variety of other embodiments and configurations may similarly be provided consistent with the scope and spirit of the invention. For example, the product may be formed to be placed around a joint, such as an elbow, knee, ankle, or shoulder. FIG. 3 shows an embodiment of the product 300 in the form of an insole that can be placed inside footwear (shoes, boots, etc.), where the product 300 provides cool or cold compresses. Note that other embodiments may be configured in the form of a footie or other form for applying cold compression to the foot.

Some embodiments are directed to a cooling apparatus that comprises a first layer comprising a liquid-absorbent sheet and a second layer, the first layer and the second layer being substantially coextensive in size, wherein one of the first layer and the second layer is configured to be applied directly to a selected body part, and wherein the first layer and the second layer are configured in a headwear shape for applying a cold compression effect to a forehead region.

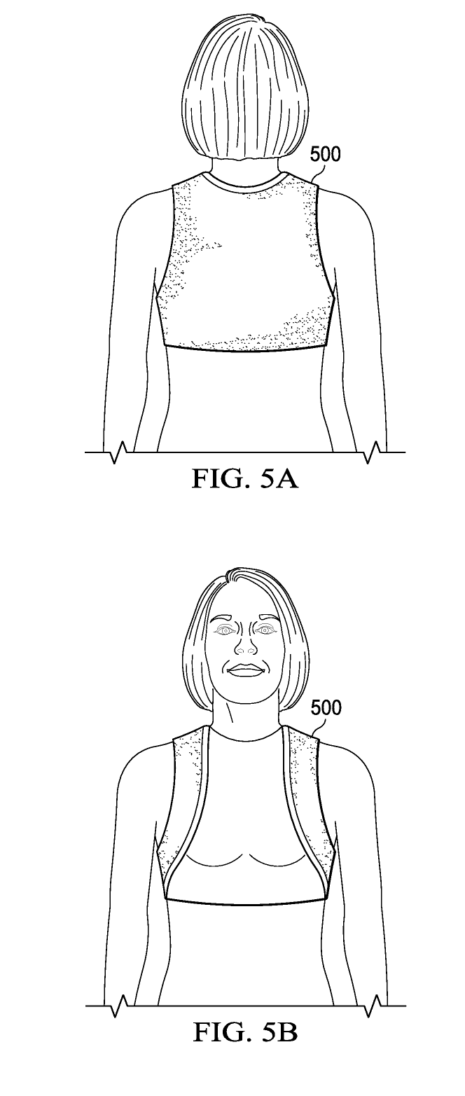

FIG. 4A shows an embodiment of the product 400 in the form of a visor, where the liquid-absorbent material is applied to the forehead of the individual. FIG. 4B shows an embodiment of the product 402 in the form of a beanie or skull cap, where the skull cap may be configured to be tight fitting to the head similar to a swim cap design. FIGS. 5A and 5B show an embodiment of the product 500 in the form of a vest or reverse-halter top, wherein straps are provided in the front, with the cooling material being in contact with the person's back to help cool the back muscles.

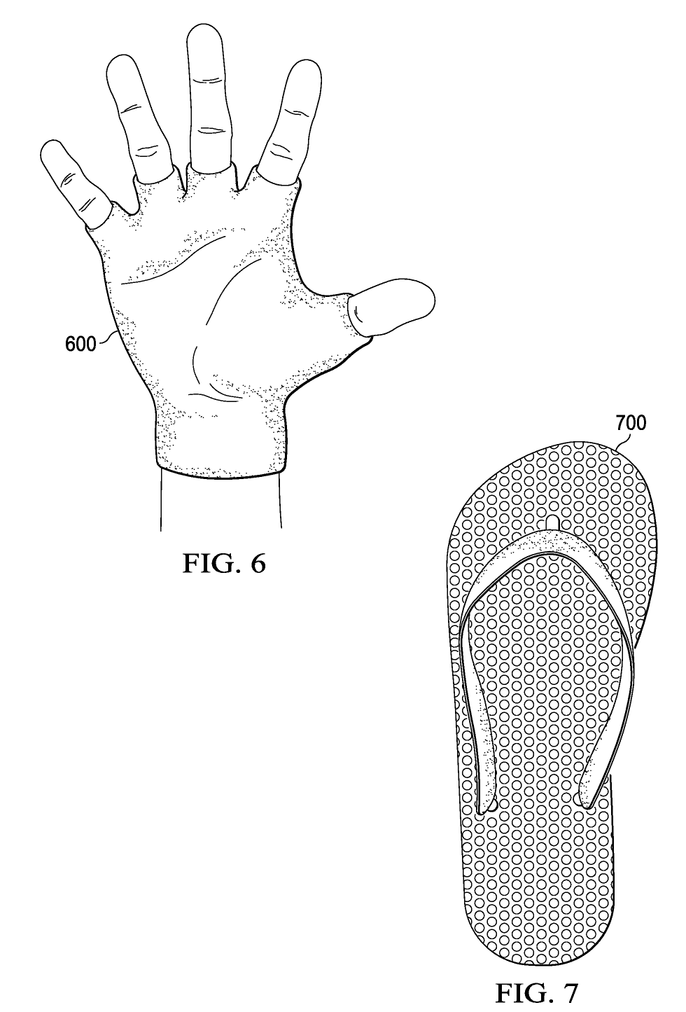

Some embodiments are directed to a therapeutic apparatus that comprises a first layer comprising a liquid-absorbent sheet a second layer, the first layer and the second layer being substantially coextensive in size, wherein one of the first layer and the second layer is configured to be applied directly to a selected body part, and wherein the first layer and the second layer are configured in a shape substantially conforming with the selected body part to receive a cold compression effect. FIG. 6 shows the product 600 in the form of a glove, thereby providing cooling to the hand and wrist in accordance with various embodiments of the present disclosure.

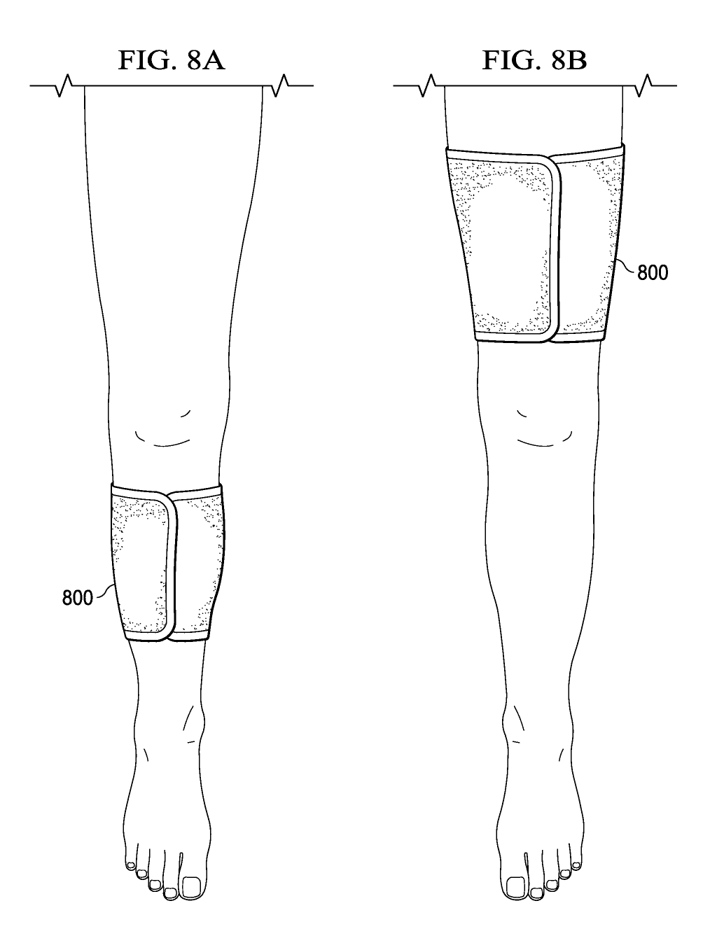

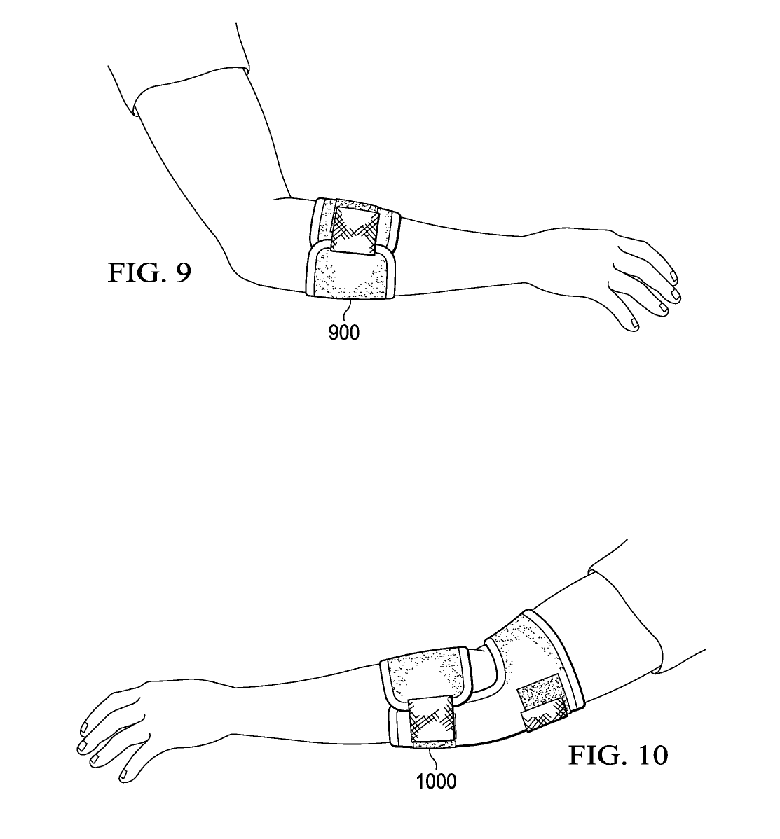

FIG. 7 shows the product 700 in the form of a sandal or flip-flop in accordance with various embodiments of the present disclosure. FIG. 8A shows the product 800 in a form that can readily be applied to the shin as a wrap, while FIG. 8B shows the product 800 being applied to the thigh. FIG. 9 shows the product 900 in the form of a forearm wrap in accordance with various embodiments of the present disclosure. For some embodiments, the product may be configured to form a wrist wrap. FIG. 10 shows the product 1000 in the form of an elbow wrap in accordance with various embodiments of the present disclosure.

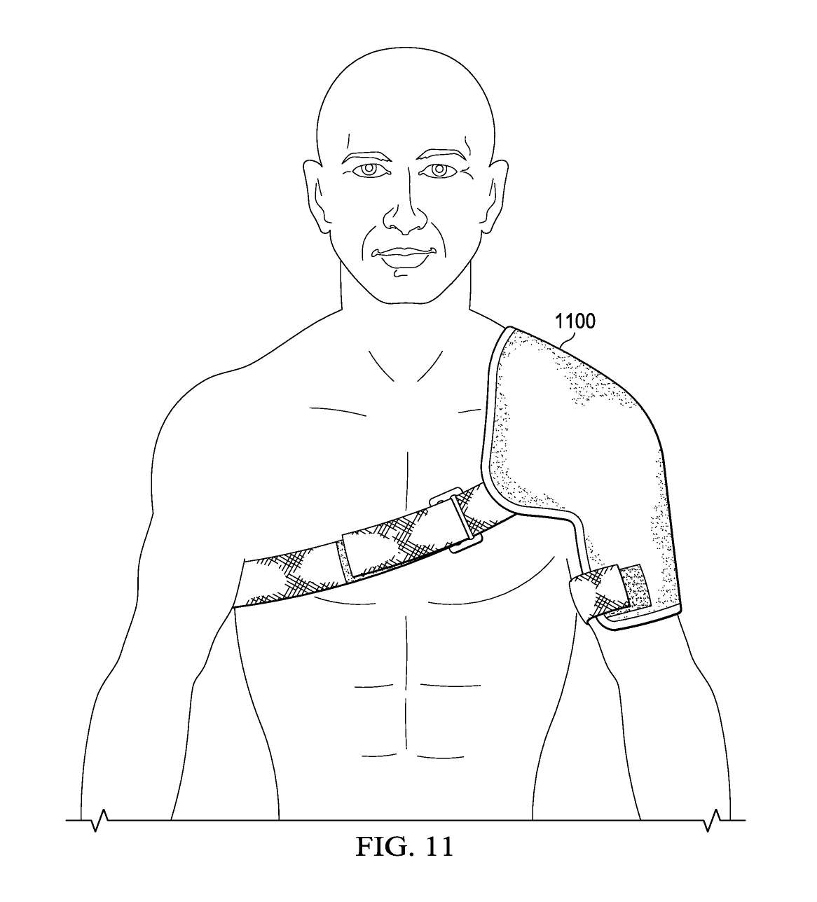

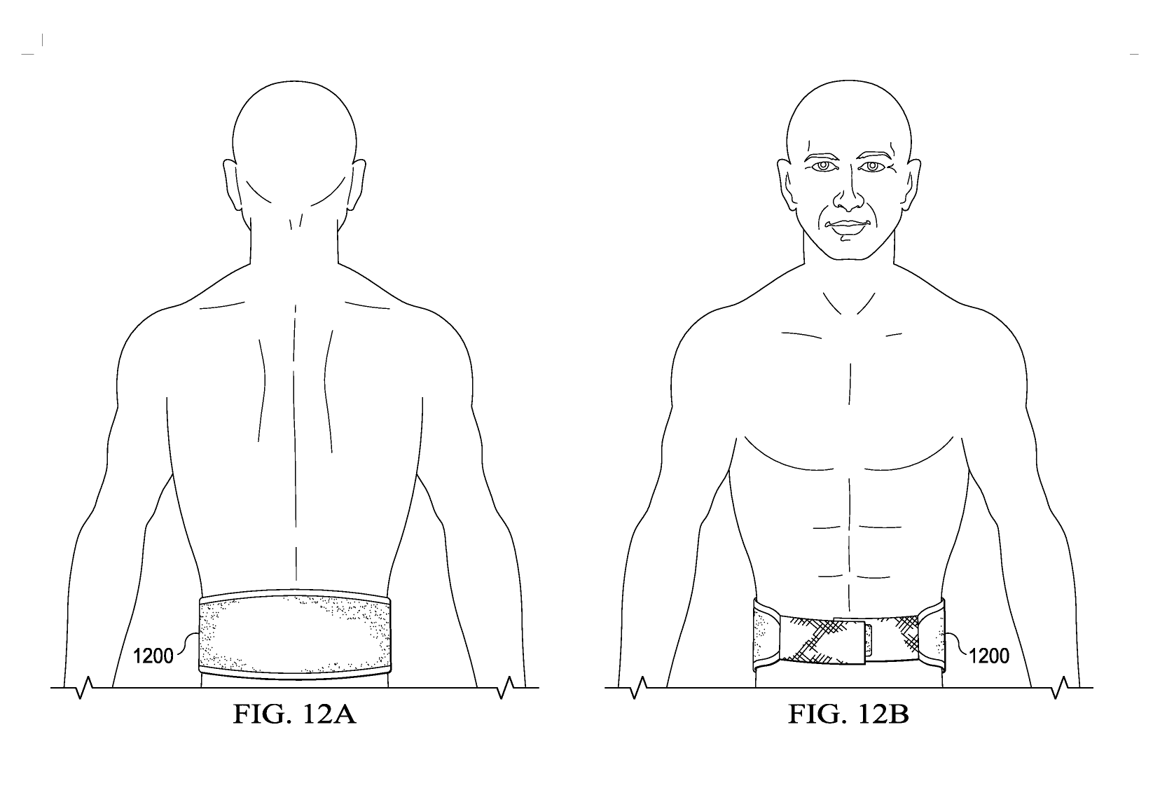

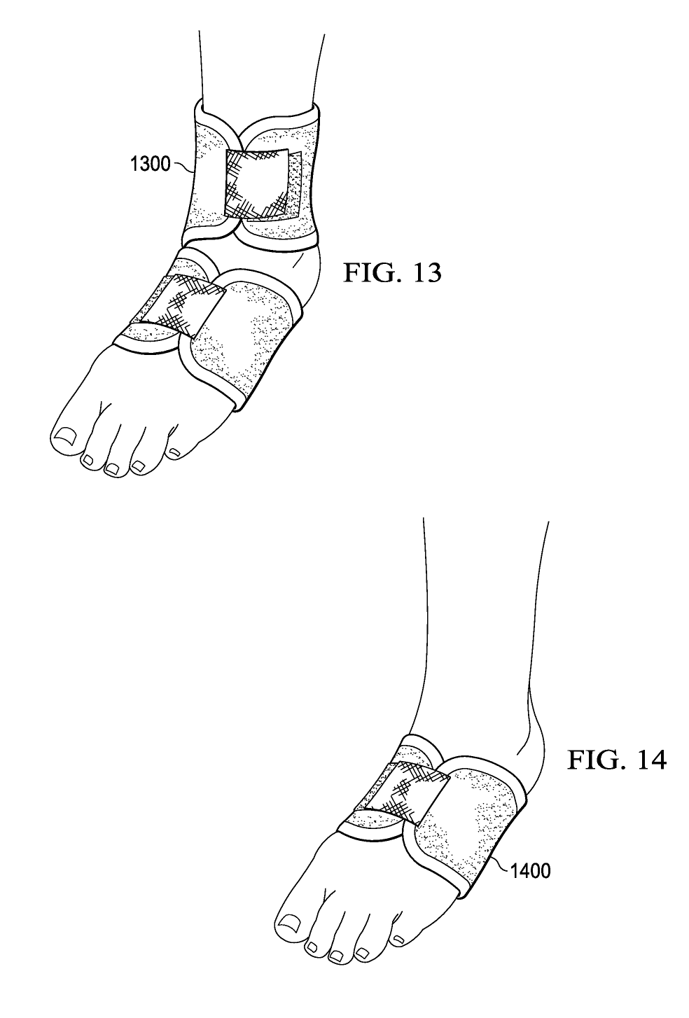

FIG. 11 shows the product 1100 in the form of a shoulder wrap in accordance with various embodiments of the present disclosure. FIGS. 12B and 12B show the product 1200 in a form suitable for a lower back wrap in accordance with various embodiments of the present disclosure. For some embodiments, the product may be configured as a hip wrap with elastic bands across the buttocks and the front. For such embodiments, the PVA material may be constructed in the form of side panels to fit each hip. The product would be pulled across the hip in manner similar to the lower back wrap in FIGS. 12A and 12B. Generally, the PVA material would not extend across the buttocks.

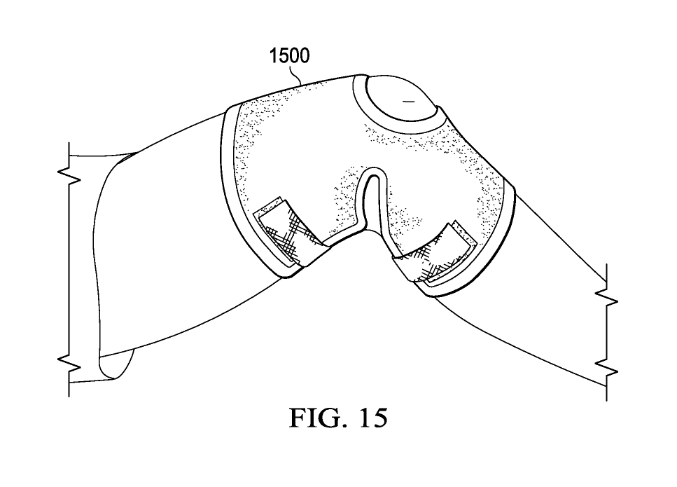

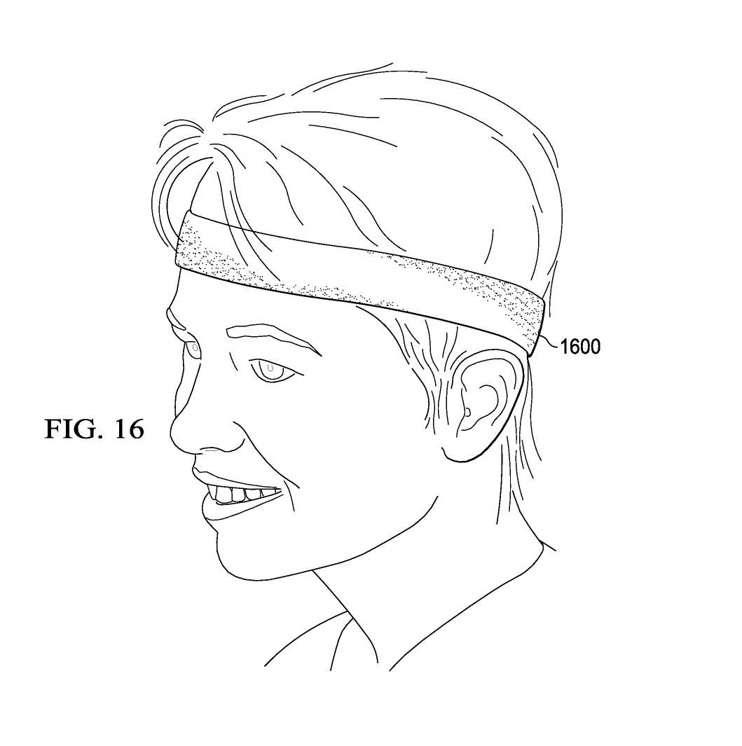

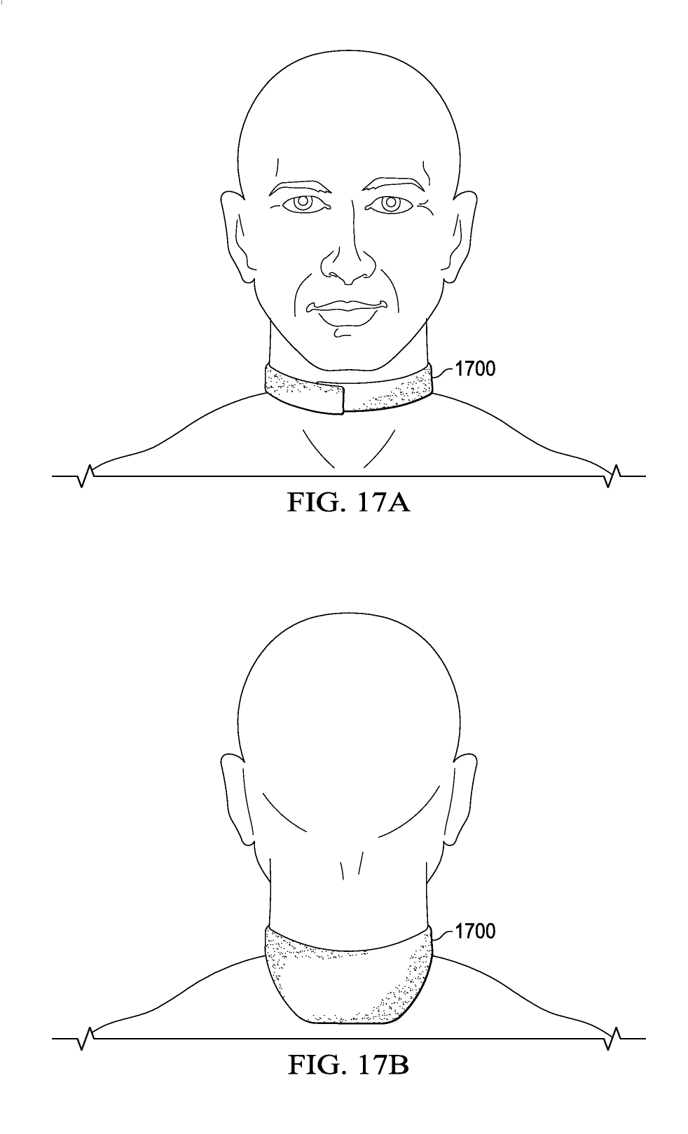

FIG. 13 shows the product 1300 in the form of an ankle wrap in accordance with various embodiments of the present disclosure. FIG. 14 shows the product 1400 in the form of a foot wrap in accordance with various embodiments of the present disclosure. FIG. 15 shows the product 1500 in the form of a knee wrap in accordance with various embodiments of the present disclosure. FIG. 16 shows the product 1600 in the form of use as a headband in accordance with various embodiments of the present disclosure. FIGS. 17A and 17B show the product 1700 in the form of use as a neckband in accordance with various embodiments of the present disclosure.

Embodiments of the product are formed from materials that permit the product to provide a cold/cool compress that retains a low temperature for hours. Furthermore, the product is made from a material that may be reused a number of times. In accordance with various embodiments, the PVA material may be soaked in water, wherein excess water may be wrung out. The moisture retained in the product will, over a period of hours, evaporate, leaving the product feeling moist and cool when applied to a body part.

In this state, the product provides various therapeutic uses, and may be used in many instances in place of cold packs. For example, in circumstances when athletes ice down after a workout or competition, the product of the present invention may be used instead. Notably, in contrast to ice treatment, the product may be applied to one or more body parts over extended periods of time, thereby aiding in recovery from injuries. Note, however, that the product is not limited to therapeutic uses and may generally be used to provide a cooling effect when desired.

For some embodiments, an initial method of activating the product comprises submerges the product in warm water to remove excess dies from the materials. Subsequent usage only requires that the product be submerged in cool (or cold) water. As described above, keeping the cool sleeve or wrap wet will allow the product to operate through evaporation, and continue cooling for hours. Further, the materials may be reused over and over by simply re-wetting and repeating the above-described process.

The cooling effect of the product on an injured or inflamed portion of the body will benefit the user in three significant ways. First, pain and swelling in the effected area of the body are reduced. Second, the product facilitates a more pain-free usage of an effected area of the body within a desired physical activity. Finally, the product also reduces the recovery time for future use of the affected body part.

The cooling effect of this material on the injured or inflamed portion of the body will benefit the user in three significant ways—1) to reduce pain and inflammation in the affected area of the body; 2) to create a more pain-free use of an effected area of the body within a desired physical activity; 3) to reduce the recovery time period of the effected body part. The areas of the body that would benefit from these products could be, but are not limited to, the feet, ankles, shins, knees, thighs, hands, wrists, forearms, elbows, shoulders, lower and upper back, neck, and head.

There are many possible environments which could benefit from the use of such applications. These would include, but are not limited to, the average retail consumer with any short term or chronic physical ailment, professional rehabilitation centers, elderly care facilities, athletes and athletics teams of all types and at all levels, Chiropractic care, medical services (with FDA approval), and any person who would benefit from cooling the body down in a hot environment.

The method of activating the cooling effect of this material is to initially submerge it in warm water and expose it to the air. Subsequent usage of the product only requires that it be submerged in cool water. Once the cool wrap, sleeve, or cap is wet for one to two minutes it becomes relatively dry, yet cool to the touch. The moisture absorbed inside the material immediately begins to evaporate and cool down whatever body part the product touches.

The length of the cooling sensation can last for several hours, depending upon the conditions of the environment. If the cool wrap, sleeve, or cap begins to dry out, you simply re-wet in cool water, wring it out, and place it back on the body to continue the cooling effect. These products can last for a very long time when properly rolled up and placed back into their original plastic tube in a cool environment for storage. To clean the products, just hand wash in warm water with liquid detergent and wring out the excess water.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

At least the following is claimed:

1. An adjustable wrap configured as a sleeve with a variable peripheral size, comprising:
a first elongated layer comprising a liquid-absorbent sheet;
a second elongated layer, the first elongated layer and the second elongated layer being substantially coextensive in size, wherein one of the first elongated layer and the second elongated layer is configured to be applied directly to a selected body part and provide a cold compression effect to the body part; and
a fastener coupled to one of the first elongated layer and the second elongated layer, the fastener comprising a first section and a second section for providing an adjustable peripheral size of the wrap.

2. The wrap of claim 6, wherein the second elongated layer comprises the liquid-absorbent sheet.

3. The wrap of claim 6, wherein the second elongated layer comprises a tricot fabric material, and wherein the second elongated layer is configured to be applied directly to the selected body part.

4. The wrap of claim 1, wherein the liquid-absorbent sheet comprises a Poly Vinyl Alcohol (PVA) based material.

5. The wrap of claim 1, wherein the fastener comprises a hook-and-loop fastener, wherein the first section comprises a hook section and the second section comprises a loop section, wherein coupling of the first and second sections define the peripheral size of the wrap.

6. The wrap of claim 1, further comprising a mesh layer disposed between the first elongated layer and the second elongated layer.

7. The wrap of claim 6, wherein the mesh comprises a substantially inelastic material.

8. The wrap of claim 6, wherein the mesh comprises an elastomeric material.

9. The wrap of claim 6, wherein the mesh is substantially coextensive in size with the first elongated layer and the second elongated layer.

10. The wrap of claim 1, wherein the fastener comprises a reusable adhesive fastener, wherein coupling of the first and second sections define the peripheral size of the wrap.

11. A therapeutic apparatus, comprising:
a first layer comprising a liquid-absorbent sheet;
a second layer, the first layer and the second layer being substantially coextensive in size,
wherein one of the first layer and the second layer is configured to be applied directly to a selected body part,
wherein the first layer and the second layer are configured in a shape substantially conforming with the selected body part to receive a cold compression effect.

12. The apparatus of claim 11, wherein the body part comprises one of: a hand; a foot; a shoulder; and an upper torso region.

13. The apparatus of claim 11, wherein the second layer comprises the liquid-absorbent sheet.

14. The apparatus of claim 13, wherein the liquid-absorbent sheet comprises a Poly Vinyl Alcohol (PVA) based material.

15. The apparatus of claim 11, further comprising a mesh layer disposed between the first layer and the second layer.

16. The wrap of claim 11, wherein the second layer comprises a tricot fabric material.

17. The wrap of claim 16, wherein the second layer is configured to be applied directly to the selected body part.

18. A cooling apparatus, comprising:
a first layer comprising a liquid-absorbent sheet;
a second layer, the first layer and the second layer being substantially coextensive in size,
wherein one of the first layer and the second layer is configured to be applied directly to a selected body part,
wherein the first layer and the second layer are configured in a headwear shape for applying a cold compression effect to a forehead region.

19. The apparatus of claim 18, wherein the headwear shape comprises one of: a headwear beanie, a baseball cap, a visor, and a headband.

20. The apparatus of claim 18, wherein the second layer comprises the liquid-absorbent sheet, and wherein the liquid-absorbent sheet comprises a Poly Vinyl Alcohol (PVA) based material.
